(19) United States
(12) Patent Application Publication
(10) Pub. No.: US 2006/0148752 A1
(43) Pub. Date: Jul. 6, 2006

(54) Method of treating soild tumors and leukemias using combination therapy of vitamin D and anti-metabolic nucleoside analogs

(76) Inventors: Candace S. Johnson, Amherst, NY (US); Donald L. Trump, Buffalo, NY (US)

Correspondence Address: NIXON PEABODY LLP - PATENT GROUP, CLINTON SQUARE, P.O. BOX 31051, ROCHESTER, NY, 14603-1051, US

(21) Appl. No.: 11/363,667

(22) Filed: Feb. 28, 2006

Related U.S. Application Data
(62) Division of application No. 11/093,123, filed on Mar. 29, 2005.
(60) Provisional application No. 60/557,516, filed on Mar. 29, 2004.

(51) Int. Cl. A61K 31/7072; A61K 31/7076; A61K 31/59

(52) U.S. Cl. 514/45; 514/49; 514/167

(57) ABSTRACT

The present invention relates to a method of inhibiting solid tumor cell or leukemia cell proliferation by first administering to a solid tumor cell or leukemia cell either vitamin D or a derivative thereof and subsequently administering at least one anti-metabolic nucleoside analog to the solid tumor cell or the leukemia cell. Also disclosed is a method of treating a cancerous condition. Methods of down regulating a p-AKt survival signaling pathway and modulating activity of a pro-apoptotic caspases is also disclosed.

This application is a divisional application of U.S. patent application Ser. No. 11/093,123, filed Mar. 29, 2005, which claims benefit of U.S. Provisional Patent Application Ser. No. 60/557,516, filed Mar. 29, 2004, each of which is hereby incorporated by reference in its entirety.

The present invention was made at least in part with funding received from the National Institutes of Health under grants CA67267, CA95045 and CA85142. The U.S. government may retain certain rights in this invention.

### FIELD OF THE INVENTION

The present invention relates to inhibiting solid tumor cell or leukemia cell proliferation, treating cancerous conditions, downregulating p-Akt survival signaling pathway, and modulating activity of a pro-apoptotic caspase.

### BACKGROUND OF THE INVENTION

Combating the growth of neoplastic cells and tumors has been a major focus of biological and medical research. Such research has led to the discovery of novel cytotoxic agents potentially useful in the treatment of neoplastic disease. Examples of cytotoxic agents commonly employed in chemotherapy include anti-metabolic agents interfering with microtubule formation, alkylating agents, platinum-based agents, anthracyclines, antibiotic agents, topoisomerase inhibitors, and other agents.

Aside from merely identifying potential chemotherapeutic agents, cancer research has led to an increased understanding of the mechanisms by which these agents act upon neoplastic cells, as well as on other cells. For example, cholecalciferol (vitamin D) can effect differentiation and reduce proliferation of several cell types both in vitro and in vivo. The active metabolite of vitamin D (1,25-dihydroxycholecalciferol (hereinafter “1,25D₃”) and analogs (e.g., 1,25-dihydroxy-16-ene-23-yne-cholecalciferol (Ro23-7553), 1,25-dihydroxy-16-ene-23-yne-26,27-hexafluoro-19-nor-cholecalciferol (Ro25-6760), etc.) mediate significant in vitro and in vivo anti-tumor activity by retarding the growth of established tumors and preventing tumor induction (Colston et al., Lancet 1:188 (1989); Belleli et al., Carcinogenesis 13:2293 (1992); McElwain et al., Mol. Cell. Diff. 3:31-50 (1995); Clark et al., J. Cancer Res. Clin. Oncol. 118:190 (1992); Zhou et al., Blood 74:82-93 (1989)). In addition to retarding neoplastic growth, 1,25D₃ induces a G₀/G₁-S phase block in the cell cycle (Godyn et al., Cell Proliferation 27:37-46 (1994); Rigby et al., J. Immunol. 135:2279-2286 (1985); Elstner et al., Cancer Res. 55:2822-2830 (1995); Wang et al., Cancer Res. 56:264-267 (1996)). These properties have led to the successful use of 1,25D₃ to treat neoplastic tumors (Cunningham et al., Br. J. Cancer 63:4673 (1991); Mackie et al., Lancet 342:172 (1993); Bower et al., Proc. Am. Assoc. Cancer Res. 32:1257 (1991)).

In addition to its antineoplastic and cell-cycle blocking effects, 1,25D₃ treatment can lead to hypercalcemia. As a result, 1,25D₃ is typically administered for therapeutic applications (e.g., metabolic bone disease) at relatively low doses (e.g., about 1 μg/day to about 2 μg/day) long term. To mitigate the effects of hypercalcemia, analogs have been developed which retain antiproliferative activity without inducing hypercalcemia (Zhou et al., Blood 73:75 (1991); Binderup et al., Biochem. Pharmacol. 42:1569 (1991); Binderup et al., page 192 in Proceedings of the 8^{th} Workshop on Vitamin D, Paris France (Norman, A. et al., Eds., Walter de Gruyter, Berlin (1991))). Many of these synthetic analogs are more potent than 1,25D₃ in inhibiting neoplastic growth (for a review of many such analogs, see Calverley et al., “Vitamin D” in Antitumor Steroids (Blickenstaff, R. T., Ed., Academic Press, Orlando (1992)).

Because of differences in the biological mechanisms of various chemotherapeatic agents, protocols involving combinations of different chemotherapeatic agents have been attempted (e.g., Jekunen et al., Br. J. Cancer 69:299-306 (1994); Yeh et al., Life Sciences 54:431-35 (1994). Combination treatment protocols aim to increase the efficacy of cytopathic protocols by using compatible chemotherapeatic agents. In turn, the possibility that sufficient antineoplastic activity can be achieved from a given combination of chemotherapeatic agents presents the possibility of reducing the dosage of individual chemotherapeatic agents to minimize harmful side effects. In part because the various chemotherapeatic agents act during different phases of the cell cycle, the success of combination protocols frequently depends upon the order of drug application (Jekunen et al., Br. J. Cancer 69:299-306 (1994); Studzinski et al., Cancer Res. 51:3451 (1991)).

There have been attempts to develop combination drug protocols based, in part, on vitamin D derivatives. For example, the inhibitory effect of concurrent combination of 1,25D₃ and platinum drugs on the growth of neoplastic cells has been studied (Saunders et al., Gynecol. Oncol. 51:155-159 (1993); Cho et al., Cancer Res. 51:2848-2853 (1991)) and similar studies have focused on concurrent combinations of 1,25D₃ and other cytotoxic agents (Tanaka et al., Clin. Orthopaed. Rel. Res. 247:290-296 (1989)). The results of these studies, however, have been less than satisfactory. In particular, the optimal sequence of drug administration has not been achieved. Moreover, the application of these approaches in therapy would require the long-term application of high doses of 1,25D₃ in some protocols, which can precipitate significant side effects.

The efficacy of pretreatment with vitamin D (or its derivatives) in combination with subsequent chemotherapeutic agent administration has been previously described in U.S. Pat. Nos. 6,087,350 and 6,559,139 to Johnston et al. The examples disclosed therein demonstrated the efficacy of vitamin D derivatives in combination with various platinum-containing agents, specifically the combinations of cisplatin and Ro23-7553, carboplatin and 1,25D₃, and paclitaxel and 1,25D₃, all of which were used to treat in vitro squamous tumor cells or in vivo induced squamous cell tumors.

The present invention is directed to extending these earlier developments to other cancerous conditions and combinations with other chemotherapeutic agents, as well as otherwise overcoming various deficiencies in the art.

### SUMMARY OF THE INVENTION

A first aspect of the present invention relates to a method of inhibiting solid tumor cell or leukemia cell proliferation. This method involves first administering to a solid tumor cell or a leukemia cell either vitamin D or a derivative thereof and subsequently administering at least one anti-metabolic nucleoside analog to the solid tumor cell or the leukemia cell. The solid tumor cell or the leukemia cell is susceptible to the first administering and the subsequent administering and proliferation thereof is inhibited.

A second aspect of the present invention relates to a method of treating a cancerous condition. This method involves first administering to a patient diagnosed with a cancerous condition selected from the group consisting of a solid tumor or a leukemia, either vitamin D or a derivative thereof and subsequently administering to the patient at least one anti-metabolic nucleoside analog. The cancer cells associated with the cancerous condition are susceptible to the first administering and the subsequent administering and progression of the cancerous condition is inhibited.

A third aspect of the present invention relates to a method of downregulating a p-Akt survival signaling pathway in a cell, particularly a cancer cell. This method involves exposing a cell exhibiting a p-Akt survival signaling pathway to a combination of (i) vitamin D or a derivative thereof and (ii) at least one anti-metabolic nucleoside analog. The exposing step reduces the activity of the p-Akt survival signaling pathway in the cell.

A fourth aspect of the present invention relates to a method of modulating activity of a pro-apoptotic caspase. This method involves exposing a cell to a combination of (i) vitamin D or a derivative thereof and (ii) at least one anti-metabolic nucleoside analog. The exposing step enhances the activity of a pro-apoptotic caspase in the cell.

The present invention demonstrates the efficacy of vitamin D (or its derivatives) in a combination therapy with anti-metabolic nucleoside drugs, particularly drugs that induce pro-apoptotic caspase activity such as the pancreatic cancer drug gemcitabine. As demonstrated in the examples, the combination of gemcitabine and calcitriol were able to synergistically activate pro-apoptotic caspase activity while decreasing anti-apoptotic (pro-survival) p-Akt pathways in an in vivo pancreatic cancer model. The demonstration of effective therapies for pancreatic cancer is, in particular, quite important because pancreatic cancer generally has a poor prognosis. Very often, diagnosis occurs only when the disease is quite advanced. Consequently, of those diagnosed, about twenty percent survive one year and fewer than five percent survive five years. Thus, a preferred embodiment of the present invention provides a significant improvement in pancreatic cancer therapy.

### BRIEF DESCRIPTION OF THE DRAWINGS

FIG. 1 is an isobologram graph illustrating combination drug effects of calcitriol and gemcitabine on capan-1 cells. Mutually exclusive CI plots were generated for gemcitabine/calcitriol combination in capan-1 cells as determined by MTT assay. Each CI was calculated from the affected fraction at each drug ratio. CI<1 indicates synergy, CI=1 indicates additivity, CI>1 indicates antagonism.

FIG. 2 is a graph showing calcitriol and gemcitabine effect on capan-1 tumor growth in nude mice. Capan-1 tumor bearing nude mice were either untreated (●), treated with calcitriol alone (2.5 μg/mouse/twice a week □, 2.5 μg/mouse/week ▪) or gemcitabine alone (6 mg/mouse/week ▴), and were treated with the two-drug combination (calcitriol 2.5 μg/twice a week+gemcitabine ● and calcitriol 2.5 μg/week+gemcitabine ♦). Tumor measurements were made 3×/week. Tumor volume=(length×width²)/2.

FIGS. 3A-F are graphs showing induction of apoptosis by calcitriol/gemcitabine in capan-1 cells (annexin V-PE assay). Capan-1 cells were pre-treated with either vehicle or 0.75 μM calcitriol for 24 h and then were treated with gemcitabine 6.25 μM or 12.5 μM for 24 h. Cells were harvested by trypsinization. Flow cytometric analysis of annexin V-PE and 7-AAD binding in capan-1 cells was performed (10,000 events were counted). The two drug combination caused an increase in early and late apoptosis as compared to each drug alone.

FIG. 4 is an immunoblot showing inhibition of survival signaling pathways in capan-1 by treatment with gemcitabine and 1,25D₃. Capan-1 cells were treated 24 h with either vehicle or 0.75 μM calcitriol and then were treated with varying concentrations of gemcitabine for another 24 h. Cells were processed for immunoblotting to assess P-ErK1/2 and P-Akt expression. P-Erk1/2 was not affected by either agent alone or by the calcitriol/gemcitabine combination. P-Akt levels were little affected by gemcitabine alone, modestly reduced by calcitriol alone, and strongly reduced by the combination of gemcitabine and calcitriol.

FIG. 5 is a series of Western blots illustrating the effects of treatment with calcitriol and gemcitabine on caspase pathway induction in capan-1 cells. Capan-1 cells were pre-treated with either vehicle 0.75 μM calcitriol for 24 h and then were treated with varying concentrations of gemcitabine. Cell lysates were prepared and analyzed by Western blot for caspase-3, caspase-6, caspase-8, and caspase-9. Caspase-8, caspase-6, and caspase-9 were not cleaved at detectable levels when cells were treated with calcitriol alone.

FIGS. 6A-D show quantitation of caspase-3, caspase-6, caspase-8, and caspase-9. Activity of caspase-3 (FIG. 6A), caspase-6 (FIG. 6B), caspase-8 (FIG. 6C), and caspase-9 (FIG. 6D) in capan-1 cells was measured by caspase-family colorimetric substrate assays, according to the manufacturer's protocol. Absorbance at 400 nm was determined, and the caspase activity was expressed as absorbance (ODU) per milligram of protein per reaction.

FIG. 7 is a photograph illustrating capan-1 tumor cells (3×10⁶ cells) inoculated subcutaneous into nude mice. At day 8-9 post implantation, when the tumors were palpable (6.5×5 mm), animals were then treated with ip calcitriol or gemcitabine alone or in combination. Tumor growth was assessed by measuring tumor size with calipers three times/week.

FIG. 8 is a photograph of tumors removed from nude mice after treatment with calcitriol or gemcitabine alone or in combination.

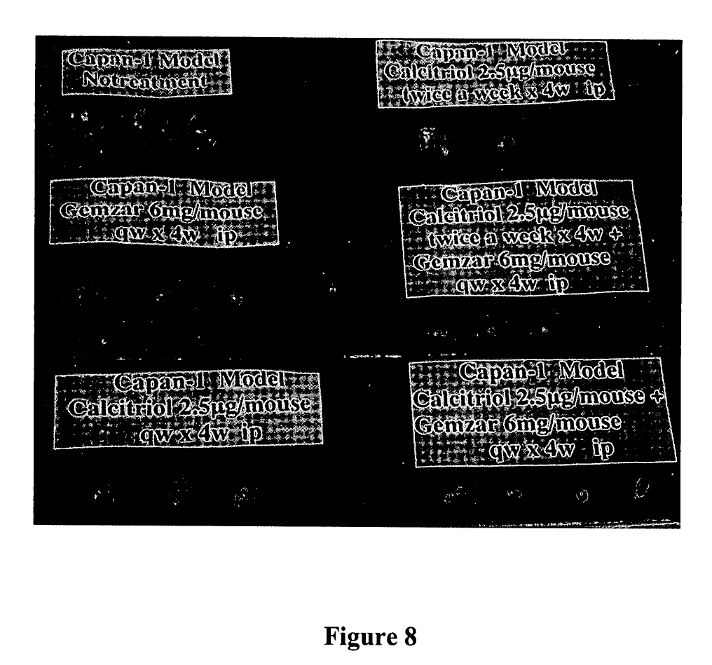

FIG. 9 is a schematic of the molecular effect of calcitriol/gemcitabine combination on survival and stress signaling pathways.

### DETAILED DESCRIPTION OF THE INVENTION

The present invention affords methods of killing a cell (e.g., a targeted cell) by the combined administration of vitamin D or a derivative thereof and at least one anti-metabolic nucleoside analog. Preferably, the administration of vitamin D or a derivative thereof occurs first, as a pretreatment of the cell. Subsequent to the pretreatment, administration of the at least one anti-metabolic nucleoside analog can occur.

When employed, any period of pretreatment can be utilized in the present invention; the exact period of pretreatment will vary depending upon the application for the inventive method. For example, in therapeutic applications, such pretreatment can be for as little as about a day to as long as about 5 days or more; more preferably, the pretreatment period is between about 2 and about 4 days (e.g., about 3 days). Persons of skill in the art are readily able to optimize the pretreatment schedule to enhance efficacy of subsequent chemotherapeutic delivery.

The cell can be solitary and isolated from other like cells (such as a single cell in culture or a metastatic or disseminated neoplastic cell in vivo), or the cell can be a member of a collection of cells (e.g., within a tumor). Preferably, the cell is a neoplastic cell (e.g., a type of cell exhibiting uncontrolled proliferation, such as cancerous or transformed cells). Neoplastic cells can be isolated (e.g., a single cell in culture or a metastatic or disseminated neoplastic cell in vivo) or present in an agglomeration, either homogeneously or, in heterogeneous combination with other cell types (neoplastic or otherwise) in a tumor or other collection of cells. Where the cell is within a tumor, the present invention provides a method of retarding the growth of the tumor by first administering vitamin D (or a derivative) to the tumor and subsequently administering the anti-metabolic nucleoside analog to the tumor.

Preferred cancer cell types include both cells of solid tumors and leukemia cells. Preferred cancers that can be treated include various leukemias, such as acute leukemias, non-Hodgkin's lymphoma, head and neck cancers, pancreatic cancer, bladder cancer, non-small cell lung cancer, etc.

By virtue of the chemotherapeutic effect on individual cells, the inventive method can reduce or substantially eliminate the number of cells added to the tumor mass over time. Preferably, the inventive method effects a reduction in the number of cells within a tumor, and, most preferably, the method leads to the partial or complete destruction of the tumor (e.g., via killing a portion or substantially all of the cells within the tumor).

Where the cell is associated with a neoplastic disorder within a patient (e.g., a human), the invention provides a method of treating the patient by first administering vitamin D (or a derivative) to the patient and subsequently administering the at least one anti-metabolic nucleoside analog to the patient. This approach is effective in treating mammals bearing intact or disseminated cancer. For example, where the cells are disseminated cells (e.g., metastatic neoplasia), the cytopathic effects of the inventive method can reduce or substantially eliminate the potential for further spread of neoplastic cells throughout the patient, thereby also reducing or minimizing the probability that such cells will proliferate to form novel tumors within the patient. Furthermore, by retarding the growth of tumors including neoplastic cells, the inventive method reduces the likelihood that cells from such tumors will eventually metastasize or disseminate. Of course, when the inventive method achieves actual reduction in tumor size (and especially elimination of the tumor), the method attenuates the pathogenic effects of such tumors within the patient. Another application is in high-dose chemotherapy requiring bone marrow transplant or reconstruction (e.g., to treat leukemic disorders) to reduce the likelihood that neoplastic cells will persist or successfully regrow.

In many instances, the pretreatment of cells or tumors with vitamin D (or a derivative) before treatment with the at least one anti-metabolic nucleoside analog effects an additive and often synergistic degree of cell death. In this context, if the effect of two or more compounds administered together in vitro (at a given concentration) is greater than the sum of the effects of each compound administered individually (at the same concentration), then the two or more compounds are considered to act synergistically. Such synergy is often achieved with anti-metabolic nucleoside analogs able to act against cells that are actively replicating and dividing, and such anti-metabolic nucleoside analogs are preferred for use in the inventive methods.

Any anti-metabolic nucleoside analogs can be used in accordance with the present invention. Gemcitabine and cytosine arabinoside are two preferred nucleoside analogs of the present invention. Gemcitabine is particularly preferred when effecting the killing of pancreatic cancer cells, non-small cell lung cancer cells, and/or bladder cancer cells. Cytosine arabinoside is particularly preferred when effecting the killing of leukemia cells, non-Hodgkin's lymphoma cells, and head and neck cancer cells.

As demonstrated in the examples, gemcitabine in combination with vitamin D (or a derivative thereof) can activate pro-apoptotic caspase-mediate pathways while also inhibiting p-Akt pro-survival pathways. Consequently, the combination described in the examples can afford synergistic treatment of cancers susceptible to gemcitabine and other nucleoside analogs. As used herein, susceptible means that the cancers are receptive to treatment with the combination therapy. Thus, other compounds and combination therapies that similarly modulate caspase and p-Akt pathways are contemplated. Exemplary chemotherapeutic agents that are known to activate caspase-mediated pre-apoptosis pathways include, without limitation, TCF4 for colorectal cancers (U.S. Pat. No. 6,762,185 to Kahn et al.), arsenic trioxide for leukemias (U.S. Pat. No. 6,855,339 to Warrell Jr., et al.), jasmonates for leukemias (U.S. Pat. No. 6,469,061 to Flescher et al.), selenomethionine for ovarian and lung cancers (U.S. Pat. No. 6,653,278 to Miki et al.), ascorbic acid esters for pancreatic, colorectal, brain, and ovarian cancers (U.S. Pat. No. 6,638,974 to Naidu), and M. phlei cell wall (“MCC”) for leukemias (U.S. Pat. No. 6,329,347 to Phillips et al.).

In addition to anti-metabolic nucleoside analogs, other cytotoxic agents can also be used, such as anti-metabolites (e.g., 5-flourouricil (5-FU), methotrexate (MTX), fludarabine, etc.), anti-microtubule agents (e.g., vincristine, vinblastine, taxanes including paclitaxel and docetaxel, etc.), alkylating agents (e.g., cyclophasphamide, melphalan, bischloroethylnitrosurea, etc.), platinum agents (e.g., cisplatin, carboplatin, oxaliplatin, JM-216, CI-973, etc.), anthracyclines (e.g., doxorubicin, daunorubicin, etc.), antibiotic agents (e.g., mitomycin-C), topoisomerase inhibitors (e.g., etoposide, camptothecins, etc.), or other cytotoxic agents (e.g., dexamethasone). The choice of cytotoxic agent(s) depends upon the application of the inventive method. For therapeutic applications, the selection of a suitable cytotoxic agent will often depend upon parameters unique to a patient; however, selecting a regimen of cytotoxins for a given chemotherapeutic protocol is within the skill of the art.

The anti-metabolic nucleoside analogs (and other cytotoxic agents) can be administered either alone or in combination with continued administration of vitamin D (or a derivative) following pretreatment. The pretreatment procedures described in U.S. Pat. Nos. 6,087,350 and 6,559,139 to Johnson et al., each of which is hereby incorporated by reference in its entirety, are contemplated herein. While, typically, treatment ceases upon administration of the anti-metabolic nucleoside analogs (and other cytotoxic agents), it can be administered continuously for a period of time (e.g., periodically over several days) as desired.

As an alternative to vitamin D, any derivative thereof suitable for potentiating the cytotoxic effect of anti-metabolic nucleoside analogs can be used within the context of the inventive method, many of which are known in the art. One preferred derivative is its natural metabolite (1,25D₃ or calcitriol). However, many vitamin D analogs have a greater antitumor activity than the native metabolite, thus the vitamin D derivative can be such an analog of calcitriol. Furthermore, where the inventive method is used for therapeutic applications, the derivative can be a non-hypercalcemic analog of calcitriol, as such analogs reduce or substantially eliminate the hypercalcemic side effects of vitamin D-based therapy. For example, the analog can be Ro23-7553, Ro24-5531, or another analog. In some embodiments, other agents that attenuate (e.g., deactivate) MAP kinase, specifically by inducing MAPK phosphatase, can be used as equivalents of vitamin D (or a derivative).

Pursuant to the inventive method, the vitamin D (or a derivative) can be provided to the cells or tumors in any suitable manner, which will, of course, depend upon the desired application for the inventive method. Thus, for example, for in vitro applications, vitamin D (or a derivative) can be added to the culture medium (e.g., mixed initially with the medium or added over time). For in vivo applications, vitamin D (or a derivative) can be mixed into an appropriate vehicle for delivery to the cell or tumor. Thus, for systemic delivery, vitamin D (or a derivative) can be supplied by subcutaneous injection, intravenously, orally, or by other suitable means. Of course, vitamin D (or a derivative) can be provided more directly to the tumor (e.g., by application of a salve or cream comprising vitamin D (or a derivative) to a tumor, by injection of a solution comprising vitamin D (or a derivative) into a tumor, etc.).

The dose of vitamin D (or a derivative) provided to the cells can vary depending upon the desired application. In research, for example, the dose can vary considerably, as dose-response analysis might be a parameter in a given study. For therapeutic applications, because the pretreatment period can be quite brief in comparison with standard vitamin D-based therapies, higher than typical doses (as discussed above) of vitamin D (or a derivative) can be employed in the inventive method without a substantial risk of hypercalcemia. Thus, for example, in a human patient, as little as 1 μg/day of vitamin D (or a derivative), which is within the normal dosage for calcitriol, can be supplied to a patient undergoing treatment, while the maximal amount can be as high as about 20 μg/day (or even higher in some larger patients). Preferably, between about 4 μg/day and about 15 μg/day (e.g., between about 7 μg/day and about 12 μg/day) of vitamin D (or a derivative) is delivered to the patient. Typically, the amount of vitamin D (or a derivative) supplied will not be so great as to pose a significant risk of inducing hypercalcemia or provoking other toxic side effects. Hence, where non-hypercalcemic vitamin D derivatives are used, higher amounts still can be employed. Thus, 30 μg/day or more (e.g., about 40 μg/day or even 50 μg/day or more) non-hypercalcemic vitamin D derivative can be delivered to a human patient during pretreatment in accordance with the inventive method. Of course, the desired dose of vitamin D (or a derivative) will depend upon the size of the patient and the mode and timing of delivery. Vitamin D (or a derivative) can be delivered once a day, or several times a day, as desired, or it can be delivered discontinuously (e.g., every other day, or every third day). The determination of such doses and schedules is well within the ordinary skill in the art.

For in vivo application, the appropriate dose of a given anti-metabolic nucleoside analog (or other cytotoxic agent) depends on the agent and its formulation, and it is well within the ordinary skill of the art to optimize dosage and formulation for a given patient. Thus, for example, such agents can be formulated for administration via oral, subcutaneous, parenteral, submucosal, intraveneous, or other suitable routes using standard methods of formulation. For example, gemcitabene can be administered from about 100 mg/m² to about 1500 mg/m² depending on a particular course of treatment, and cytosine arabinoside can be administered to achieve concentrations ranging from about 100 mg/m² to about 2-3 g/m² (high dose). Other cytotoxic agents, if administered, can be utilized according to known dosage regimes.

In all aspects of the invention that involve in vivo application, preferably the method is employed to minimize the hypercalcemic properties of vitamin D. One manner of accomplishing this is to employ a nonhypercalcemic analog. Alternatively, or in conjunction with the use of such analogs, an agent that mitigates hypercalcemia can be adjunctively delivered to the patient. While any such agent can be employed, bisphosphonates (e.g., alendronate, clodronate, etidronate, ibandronate, pamidronate, risedronate, tiludronate, zoledronate, etc.) are preferred agents for adjunctive administration. Such agents can be administered in any suitable manner to mitigate hypercalcemia. Thus, they can be formulated into suitable preparations and delivered subcutaneously, intravenously, orally, etc., as appropriate. Also, such agents can be administered concurrently, prior to, or subsequent to vitamin D (or a derivative). The dosage of such agents will, of course, vary with the potency of the compounds and also to mitigate any unwanted side effects. Thus, for example, for administration to human patients, the dosage of bisphosphonates can vary between about 1 mg/day and 500 mg/day (e.g., between about 5 mg/day and 100 mg/day), such as between about 10 mg/day and about 50 mg/day, or even between about 30 mg/day and about 40 mg/day, depending on the potency of the bisphosphonates. Generally, it is preferred to employ a more potent bisphosphonate, as less of the agent need be employed to achieve the antihypercalcemic effects. Thus, a most preferred bisphosphonate is zoledronate, as it is effective even at very low doses (e.g., between about 0.5 mg/day and about 2 mg/day in human patients, or between about 5 μg/kg to about 25 μg/kg body weight).

In addition, a glucocorticoid (e.g., cortisol, dexamethasone, hydrocortisone, methylprednisolone, prednisolone, prednisone, etc.), diphenhydramine, rantidine, antiemetic-ondasteron, or ganistron can be adjunctively administered, and such agents can be administered with vitamin D (or a derivative).

In addition to the foregoing, applicants have identified mechanisms of action for preferred regimen of the present invention. In particular, the combination of anti-metabolic nucleoside analogs and vitamin D (or a derivative) can be used to both reduce the activity of the p-Akt survival signaling pathway in a targeted cell (or tumor) and enhance activity of a pro-apoptotic caspases in the targeted cell. As demonstrated in the following examples, p-Akt activity was disrupted and the activity of caspase-6, caspase-8, and caspase-9 (among other caspases) can be enhanced.

### EXAMPLES

The following examples are provided to illustrate embodiments of the present invention but are by no means intended to limit its scope.

Materials and Methods

Tumor Cells and Model System

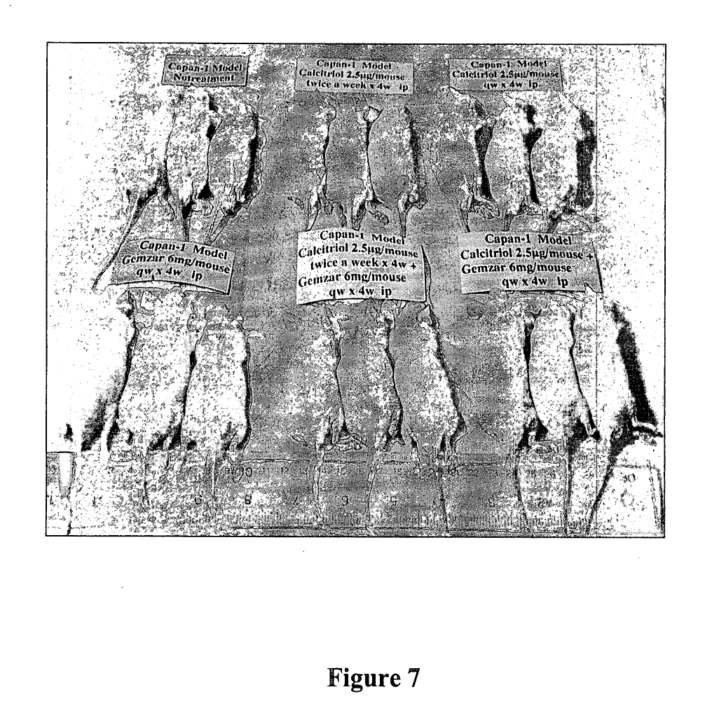

Capan-1 (human pancreas, adenocarcinoma, ATCC) was thawed and freshly cultured for all of the in vitro and in vivo studies. Capan-1 tumors were routinely produced by subcutaneous inoculation of 3×10⁶ log-phase tissue culture cells in the right rear flank of the animals (FIG. 7).

Reagents

Calcitriol (1,25-dihydroxycholecalciferol) (Hoffmann-LaRoche, Inc., Nutley, N.J.) was reconstituted in 100% ethyl alcohol, and stored protected from light under a layer of nitrogen gas at −700° C. Dilutions of calcitriol were made in medium just prior to use. Gemcitabine (Gemzar, Eli Lilly Co., Indianapolis, Ind.) was also diluted in medium just prior to use.

In Vitro Cytotoxicity Assay

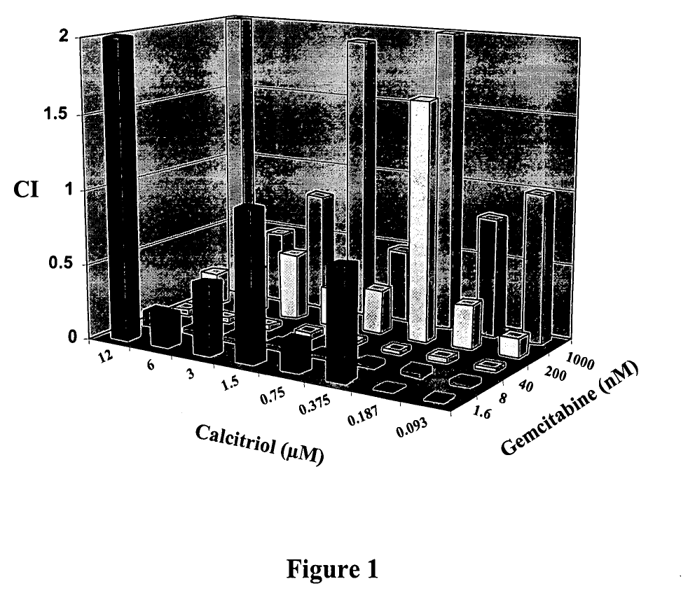

Capan-1 cells were suspended at 0.15×10⁵ cells/ml, and 100 μl/well were dispensed into 96 well microtiter plates. The following day, various concentrations of each agent were added. The cells were harvested 48 h after treatment by adding 20 μl of a stock solution of 0.5% MTT (5 mg/ml) to each well. The plates were incubated for an additional 2-3 h at 37° C. Formazan crystals were dissolved with 100 μl of 10% SDS/10 mM HCl solution overnight at 37° C. The absorbance was measured using an ELISA plate reader at a wavelength of 540 nm. The CalcuSyn program (T. C. Chou and M. P. Hayball, Biosoft®) was used to analyze the drug combinations (FIG. 1). Constant ratios of drug concentrations were used in these studies and mutually exclusive equations were used to determine the combination index (“CI”). CI<1 indicates synergistic effects, CI=1 indicates additive effects and CI>1 indicates antagonistic effects.

Tumor Growth Assay

Capan-1 tumor cells (3×10⁶ cells) were inoculated subcutaneous into nude mice (FIG. 7). At day 8-9 post implantation, when the tumors were palpable (6.5×5 mm), animals were then treated with ip calcitriol or gemcitabine alone or in combination. Tumor growth was assessed by measuring tumor size with calipers three times/week. Tumor volumes were calculated by (length×width²)÷2 and expressed as a fraction of pre-treatment size at the time of the first treatment.

Quantitation of Caspase-3, Caspase-6, Caspase-8, Caspase-9 Activity

Caspase-3, 6, 8, and 9 activity was measured using the caspase-family Colorimetric Assay kit from R&D Systems (Minneapolis, Minn.), according to the manufacturer's protocol. Caspase activity was expressed as absorbance (O.D.) per milligram of protein per reaction.

Flow Cytometric Analysis

Capan-1 cells were treated for 48 h and harvested by trypsinization. Cells were stained with Annexin V-PE and 7-AAD according to the manufacturer's instructions (BD Pharmingen, San Diego, Calif.).

Western Blots

For Western blot analysis, capan-1 cells were harvested and lysed in Triton-X100/SDS buffer as described previously (Perrais et al., J. Biol. Chem. 276(33):30923-33 (2001), which is hereby incorporated by reference in its entirety). Proteins were transferred to PVDF membrane, and Western blots developed using Renaissance Enhanced Chemiluminescence Reagents (NEN Life Science Products, Boston, Mass.) or SuperSignal (Pierce, Rockford, Ill.).

### Example 1

### Treatment of Induced Pancreatic Cancer with Gemcitabine and Calcitriol in Combination

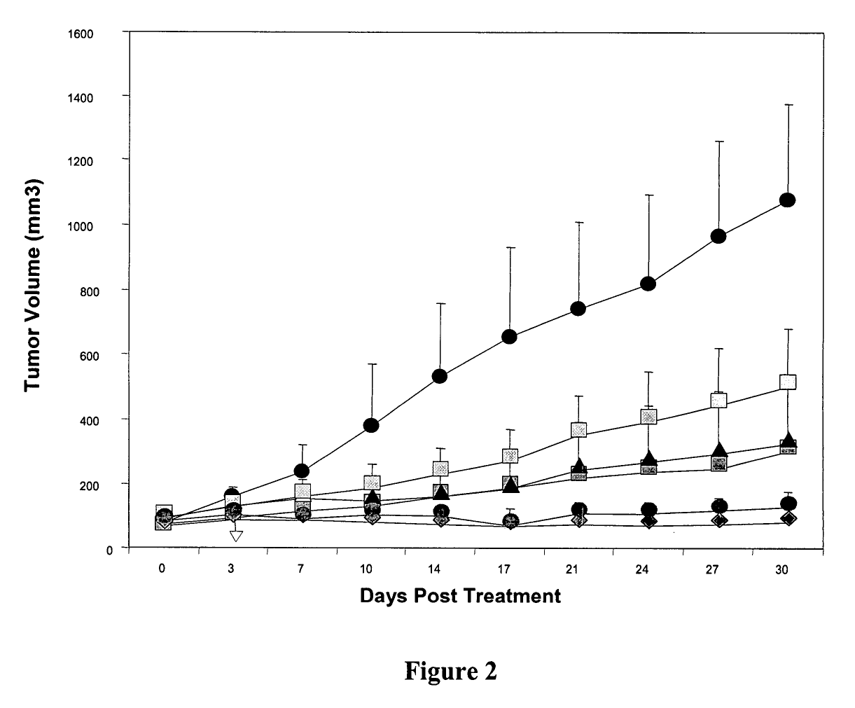

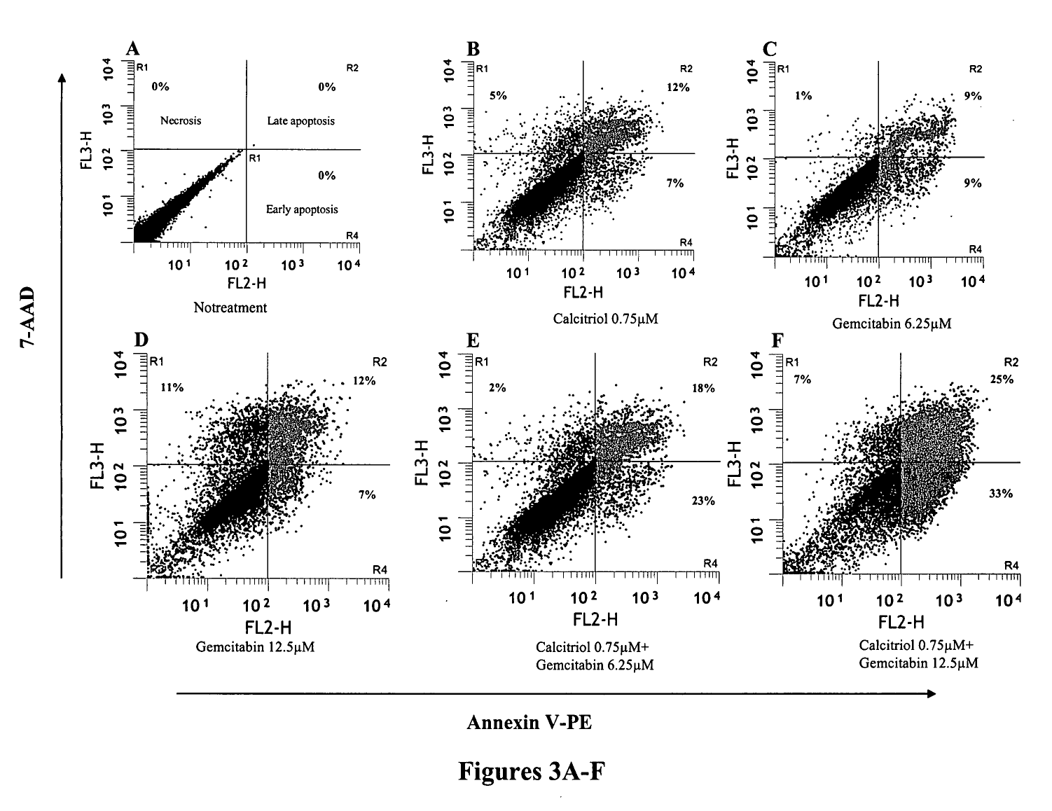

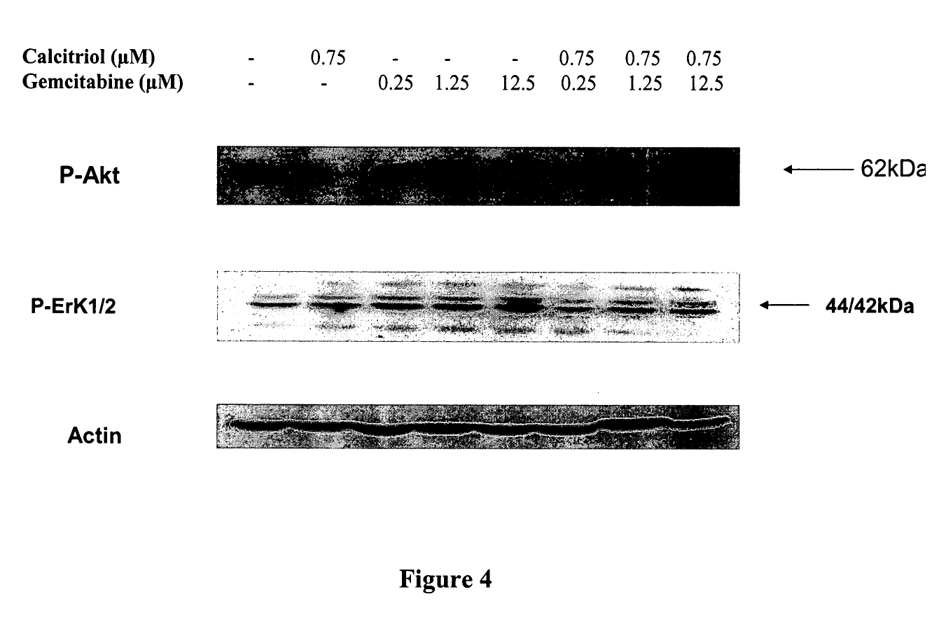

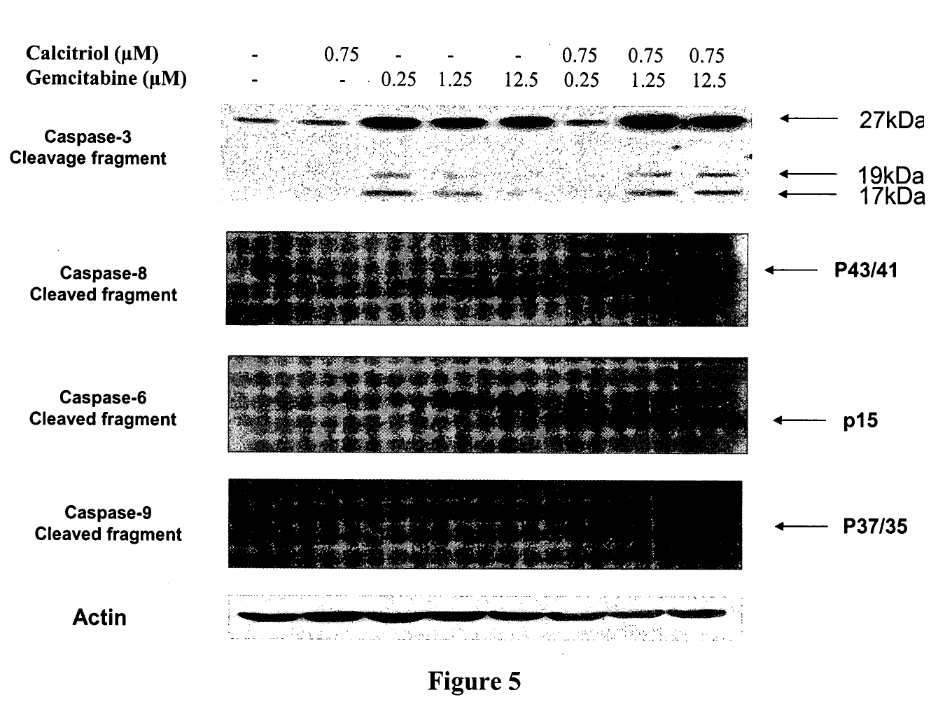

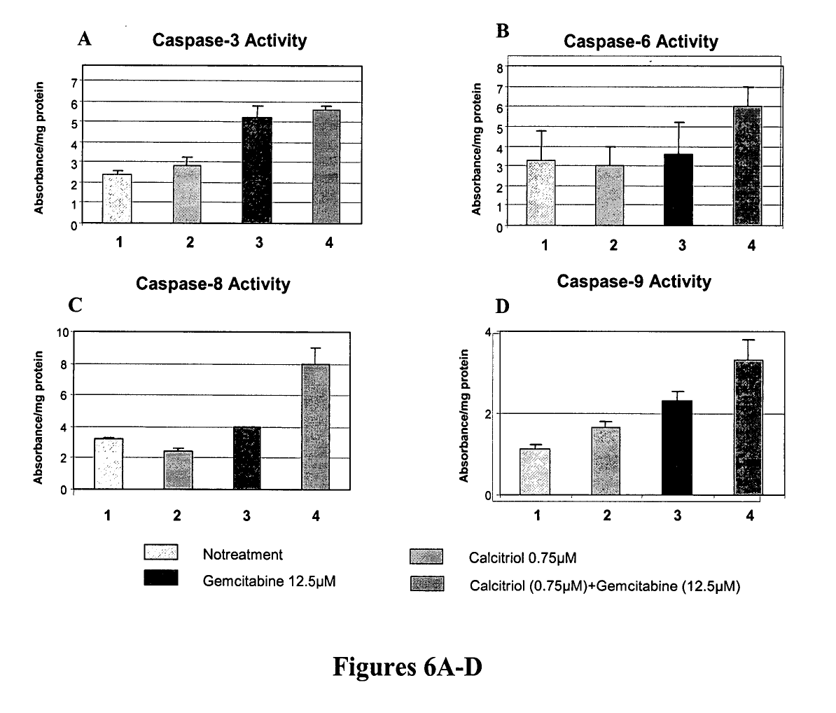

The chemotherapeutic agent most commonly used to treat metastatic cancer of the pancreas is gemcitabine. It was therefore investigated whether calcitriol, as an exemplary vitamin D derivative, could potentiate the cytotoxic activity of gemcitabine using the human pancreatic cancer cell line (capan-1). Isobologram analysis revealed that synergy was observed over a wide range of drug concentrations (FIG. 1). Tumor regrowth delay studies were performed using subcutaneous implantation of capan-1 cells in nude mice. As shown in FIG. 2, calcitriol in combination with gemcitabine produced a significant reduction of capan-1 tumor volume compared to control and either agent alone (p<0.01). By flow cytometric assay, treatment with the calcitriol/gemcitabine combination resulted in an increase in apoptosis in capan-1 cells over that seen with either agent alone (FIGS. 3A-F). To study the molecular events resulting in the enhanced cytotoxicity observed for this combination, cells were processed for immunoblotting and expression of P-Akt and P-Erk1/2 assessed. Levels of the pro-survival signaling molecule P-Akt were not affected by gemcitabine alone, modestly reduced by the calcitriol alone, and strongly reduced by the combination of both agents (FIG. 4). P-Erk1/2 expression was not affected by either agent alone or by the calcitriol/gemcitabine combination (FIG. 4). These data suggest that the synergistic cytotoxicity observed for this combination may result from strong inhibition of the P-Akt survival signaling pathway. Activation of the initiator caspases, caspase-8 and caspase-9 was also examined. Gemcitabine produced an increase in the cleaved, activated form of caspase-8 (p43/p41) in capan-1 cells across all drug concentrations (FIG. 5). However, there was no change in caspase-8 activation by calcitriol (FIG. 6C). In contrast, the cleavage/activation of caspase-9 was increased by calcitriol alone, but there was no significant change in activation by gemcitabine (FIG. 6D). An increase in downstream effector caspase-3 cleavage was observed with the combination as compared to either agent alone (FIG. 5). Based on the results obtained to date, gemcitabine may trigger apoptosis via initial cleavage of procaspase-8 and activation of subsequent downstream events. In the capan-1 cell line, calcitriol-induced apoptosis appears to be preferentially mediated by caspase-9. The increase in antiproliferative activity with calcitriol and gemcitabine may be attributed to the enhancement of apoptotic signaling due to loss of P-Akt.

Median dose effect and isobologram analyses reveal that the combination of calcitriol with gemcitabine is strongly synergistic in vitro. The combination also shows significantly greater in vivo activity using capan-1 cells.

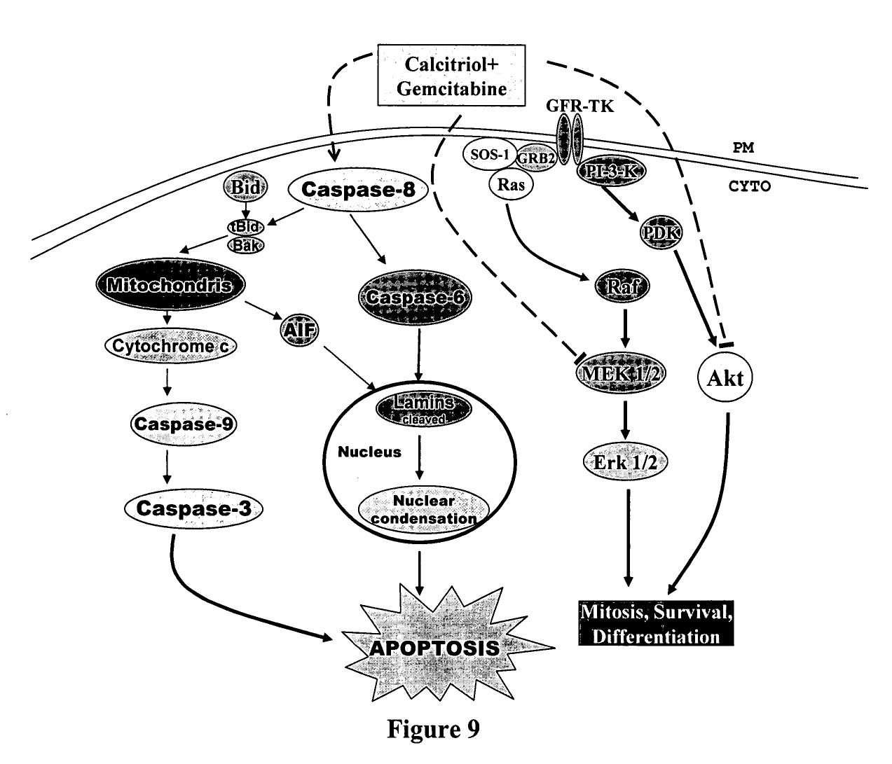

Treatment of capan-1 cells with calcitriol and gemcitabine is associated with an increase in caspase-8 cleavage and enhanced activation of caspase-6. This suggests that enhanced cytotoxicity may be mediated by up-regulation of the activation of the caspase-8/caspase-6/nuclear lamin pathway (FIG. 9).

Caspase-9 was modestly increased in capan-1 cells after treatment with calcitriol or gemcitabine alone and strongly increased by the combination. Caspase-3 was modestly increased by the combination of agents. This suggests that the synergistic cytotoxicity observed for this combination may result from the generation of active caspase-9 cleavage products. Caspase-9, in turn, activates the executioner caspases, such as caspase-3 (FIG. 9).

The level of the pro-survival signaling molecule, P-Akt, was strongly decreased by the combination of gemcitabine and calcitriol, but the expression of P-Erk1/2 was not significantly affected by the two agent combination. These data indicate that gemcitabine/calcitriol enhanced cytotoxicity may result, at least in part, from inhibition of the P-Akt survival signaling pathway (FIG. 9).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of downregulating a p-Akt survival signaling pathway comprising:
exposing a cell exhibiting a p-Akt survival signaling pathway to a combination of (i) vitamin D or a derivative thereof and (ii) at least one anti-metabolic nucleoside analog, wherein said exposing reduces the activity of the p-Akt survival signaling pathway in said cell.

2. The method according to claim 1 wherein the at least one anti-metabolic nucleoside analog is selected from the group of gemcitabine and cytosine arabinoside.

3. The method according to claim 1 wherein said exposing is carried out with a derivative of vitamin D.

4. The method according to claim 3 wherein the derivative of vitamin D is calcitriol.

5. A method of modulating activity of a pro-apoptotic caspase, said method comprising:
exposing a cell to a combination of (i) vitamin D or a derivative thereof and (ii) at least one anti-metabolic nucleoside analog, wherein said exposing enhances the activity of a pro-apoptotic caspase in said cell.

6. The method according to claim 5 wherein the at least one anti-metabolic nucleoside analog is selected from the group of gemcitabine and cytosine arabinoside.

7. The method according to claim 5 wherein said exposing is carried out with a derivative of vitamin D.

8. The method according to claim 7 wherein the derivative of vitamin D is calcitriol.

9. The method according to claim 5 wherein the pro-apoptotic caspase is selected from the group of caspase-3, caspase-6, caspase-8, caspase-9, or combinations thereof.
